(19) United States
(12) Patent Application Publication
(10) Pub. No.: US 2026/0308497 A1
(43) Pub. Date: Oct. 8, 2026

(54) MICROPHYSIOLOGICAL SYSTEM DEVICES WITH SELF-CONTAINED PHOTONIC INTEGRATED CIRCUIT SENSORS

(71) Applicant: University of Rochester, Rochester, NY (US)

(72) Inventors: Joseph BUCUKOVSKI, Rochester, NY (US); Michael BRYAN, Rochester, NY (US); Daniel J. STEINER, Rochester, NY (US); John S. COGNETTI, Stockholm (SE); Katherine A. DANIEL, Rochester, NY (US); Raquel E. AJALIK, Rochester, NY (US); Hani A. AWAD, Rochester, NY (US); James L. McGRATH, Fairport, NY (US); Benjamin L MILLER, Penfield, NY (US)

(73) Assignee: University of Rochester, Rochester, NY (US)

(21) Appl. No.: 19/481,865

(22) PCT Filed: Jun. 26, 2024
(86) PCT No.: PCT/US2024/035683

§ 371 Date: Nov. 5, 2025

Related U.S. Application Data
(60) Provisional application No. 63/523,340, filed on Jun. 26, 2023.

(51) Int. Cl. C12M 3/00; C12M 1/12; C12M 1/34; C12M 3/06

(52) U.S. Cl. CPC C12M 21/08; C12M 23/16; C12M 25/02; C12M 41/32

(57) ABSTRACT

The disclosure provides microphysiological systems (“MPS”) that have self-contained, on-board photonic integrated circuit sensors. The self-contained, on-board photonic integrated circuit sensors can be coupled from the back side of the sensors, allowing the MPS to be grouped in arrays allowing high throughput screening.

### CROSS-REFERENCE TO RELATED APPLICATIONS

This application claims priority to, and the benefit of, U.S. Provisional Patent Application No. 63/523,340, filed Jun. 26, 2023, the contents of which are incorporated herein by reference in their entirety.

### STATEMENT OF FEDERAL FUNDING

This invention was made with government support under 1UG3TR003281-01 and 4UH3TR003281-03, awarded by the National Institutes of Health. The government has certain rights in the invention.

### BACKGROUND OF THE INVENTION

For decades, scientists have studied biological functions by use of cell cultures in in vitro systems, followed by more expensive, but usually more informative, studies in animal models. Unfortunately, both cell culture systems and animal models have significant drawbacks. Cell culture systems typically use primary patient samples or cell lines of a single cell type, and by their nature are incapable of recapitulating the interactions between cell types in an organ. Animal models are not only costly, making it hard to scale them for screening, but can give information irrelevant for humans due, for example, to differences in enzymes or pathways in the animal used for the model and those present in humans. The problems presented by such systems contribute to, among other things, the loss of hundreds of millions of dollars in preclinical development and testing of potential therapeutics that then fail in clinical trials.

Over a decade ago, Huh et al. reported the development of a hybrid approach that could provide information at the level of a tissue or organ, as opposed to the cell level. See, Huh et al., Science, 2010, 328:1662-1668. Such “tissue chips” and “organs-on-a-chip” are microfabricated devices that support multicellular cultures of human cells interacting in microenvironments that more realistically resemble tissue. These tissue chip and “organ-on-a-chip” technologies (also referred to as “microphysiological systems,” or “3D cell culture”), are intended to fill some of the gaps between simple cell cultures and in vivo animal studies, addressing some of the deficiencies in both. For example, in 2015, Huh reported on developing a “lung-on-a-chip” model using “soft lithography-based microfabrication techniques to construct a compartmentalized three-dimensional microchannel system consisting of upper and lower cell culture chambers separated by a 10-μm-thick microporous elastomeric membrane made of poly-(dimethylsiloxane).” (Huh, Ann Am Thorac Soc. 2015; 12(Suppl 1): S42-S44.doi: 10.1513/AnnalsATS.201410-442MG). Human alveolar epithelial cells were seeded into the upper chamber and pulmonary microvascular endothelial cells were seeded onto the lower chamber and both types of cells were allowed to adhere to their respective side of the membrane. Huh reported that the system allowed investigation of the interplay between the different types of cells on the two sides of the membrane when one side was exposed to a stimulus, such as the introduction of proinflammatory cytokines. Id.

During the decade that tissue chips and organ-on-a-chip systems have been available, they have been explored as alternatives for simple cell culture systems. While they represent an advance over single-layer cell cultures, however, several significant deficiencies have become evident.

In particular, there exists a lack of effective methods for analyzing the response of tissue chips and organ-on-a-chip systems to changes in their environment. Analysis is currently limited largely to methods such as lysing the cells on the chip or subjecting them to immunofluorescence microscopy. Immunofluorescence-based assays, such as ELISAs, however, are irreversible by nature, meaning that once the measurement is taken, the experiment is over. Thus, deciphering time courses of analyte secretion or passage through the barrier used to constrain the cells on the chip requires many resources to repeat the experiment at each time point, increasing the time, cost, and variability of such studies.

Tracking changes to the tissues or organ disposed on a chip requires multiple chips run in parallel so that a chip is available to be subjected to an experiment-ending analysis at each time point for which information is desired. It would be desirable to be able to assess the behavior of the organ-on-a-chip in real time in a nondestructive manner. While some investigators have tried to address this problem by integrating sensors substantially downstream of the system under study, this creates its own problems by decoupling the sensor from the organ-on-a-chip both temporally and spatially. Additionally, studies of cells cultured in current tissue chips is hampered by the fact that it is difficult to access the cells themselves.

Earlier work by some of the present inventors reported the development of tissue chips that have on-board photonic integrated circuits, or “PIC.” See, e.g., International Patent Application No. PCT/US 2021/058498, published as Publication No. WO 2022/099161. This application further disclosed the use of devices bearing on-board PICs to monitor changes to tendon cells, known as tenocytes.

The need for organs-on-a-chip and tissues-on-a-chip that recapitulate aspects of biology is only increasing. As of 2023, the FDA is permitted to base an IND approval on results from methodologies, such as organs-on-a-chip, that do not use animal models. See, Wadman, M., Science, 379(6628): 127-128 (2023). Accordingly, even incremental improvements that allow better monitoring of the effects of agents on tissues or organ models can have significant value in predicting the safety and efficacy of drug candidates and whether they should be advanced into clinical trials.

It would be desirable to have methods and devices that allow assessing the effects on cells on a tissue chip or organ-on-a-chip that are non-destructive and that do not decouple sensors in time and space from the tissue chip or organ-on-a-chip. And, it would be useful to have devices and methods that afford access to cells cultured in such chips and systems. Surprisingly, the present invention fulfills these and other needs.

### BRIEF SUMMARY OF THE INVENTION

In a first group of embodiments, the invention provides microphysiological systems, having disposed within them a self-contained photonic integrated circuit sensor chip. In some embodiments, the microphysiological system is an organ-on-a-chip. In some embodiments, the microphysiological system is an tissue-on-a-chip. In some embodiments, the system has a first compartment, which first compartment comprises a first flow channel, wherein said self-contained photonic integrated circuit sensor chip is positioned in said first flow channel. In some embodiments, thelself-contained photonic integrated circuit sensor chip is fixed in position in said first flow channel. In some embodiments, the said self-contained photonic integrated circuit sensor chip is fixed in position in said first flow channel by an adhesive. In some embodiments, the self-contained photonic integrated circuit sensor chip is fixed in position in said first flow channel by a layer of said microphysiological system shaped to fit around a portion of said self-contained photonic integrated circuit sensor chip. In some embodiments, the first compartment further comprises a second flow channel. In some embodiments, the system further comprises a second compartment, which second top compartment is positioned over said first compartment. In some embodiments, the a first ultrathin membrane is positioned between said second compartment and said first compartment. In some embodiments, the a second ultrathin membrane is positioned between said second compartment and said first compartment. In some embodiments, the first ultrathin membrane is positioned between said second compartment and said first compartment over said self-contained photonic integrated circuit sensor chip. In some embodiments, the a plurality of cells of a first cell type are seeded on said ultrathin membrane, and a plurality of cells of a second cell type are seeded in said first flow channel. In another group of embodiments, the invention provides systems for studying an analyte of interest released from cells in a microphysiological system device, said system comprising (a) a microphysiological system device having disposed within it a self-contained photonic integrated circuit sensor chip having photonic ring sensors, waveguides, an input grating coupler and an output grating coupler, and (b) an external apparatus for (i) coupling light from a laser to said input grating coupler of said self-contained photonic integrated circuit sensor chip through the back side of said and (ii) coupling light from said output grating coupler of said self-contained photonic integrated circuit sensor chip through the back side of said self-contained photonic integrated circuit sensor chip. In some embodiments, the photonic ring sensors of self-contained photonic integrated circuit sensor chip have been derivatized to detect said analyte of interest. In some embodiments, the microphysiological system device is a tissue-on-a-chip. In some embodiments, the microphysiological system device is an organ-on-a-chip.

In another group of embodiments, the invention provides methods for studying an analyte of interest released from cells in a microphysiological system device, said methods comprising (1) providing (a) a microphysiological system device having disposed within it a self-contained photonic integrated circuit sensor chip having photonic ring sensors, waveguides, an input grating coupler and an output grating coupler, which photonic ring sensors have been derivatized to detect said analyte of interest, and (b) an external apparatus for (i) coupling light from a laser to said input grating coupler of said self-contained photonic integrated circuit sensor chip through the back side of said and (ii) coupling light from said output grating coupler of said self-contained photonic integrated circuit sensor chip through the back side of said self-contained photonic integrated circuit sensor chip, (2) growing cells in a fluid in said microphysiological system device, and (3) contacting said derivatized photonic ring sensors with said fluid in which siad cells have been grown, whereby a change in light from said derivatized photonic ring sensors indicates the presence of said analyte of interest.

In another group of embodiments, the invention provides an array of at least two microphysiological system devices having disposed within them a self-contained photonic integrated circuit sensor chip

### BRIEF DESCRIPTION OF THE DRAWINGS

FIG. 1. FIG. 1 shows an exemplar chip bearing a photonic integrated circuit. The circles etched into the surface hold contain photonic ring sensors connected by wave guides (the lines on the surface of the chip) to input and output grating couplers.

FIGS. 2A and 2B. FIG. 2A presents an embodiment of a tissue-on-a-chip device. The device has a central well in the upper (“apical”) portion, at the bottom of which is disposed a chip having two windows, each of which bears an ultrathin membrane, which is so thin as to be transparent. When media or reagents are added to the well, the membranes in the two windows are fluidly connected by the medium. The bottom of the device of FIG. 2A has a first and a second flow channel, each of which has two ports allowing fluid introduced through a first port at the top of the device to flow through the flow channel and out of the device through the second port serving the respective channel. Cells can be positioned in the bottom flow channels under the ultrathin membranes. A photonic integrated circuit (“PIC”) is visible under the upper ultrathin membrane in fluid connection with the cells in the first channel. In FIG. 2A, the circles containing the ring sensors photonic ring sensors on the photonic integrated circuit can be seen through the ultrathin membrane. FIG. 2B is a cartoon showing a typical use of the device of FIG. 2A. In this embodiment, epithelial cells (“ECs”) are depicted growing on the upper (apical) side of the slots of the ultrathin membrane (the ultrathin membrane is depicted by horizontal dashed lines), while tenocytes are cultured in a hydrogel in the lower flow channel on the left side. Dotted lines with arrowheads depict the diffusion of factors released by the ECs into the flow channel containing the PIC, where they can contact the PIC, as shown by the generally horizontal line of dots terminating in an arrowhead.

FIGS. 3A and 3B. FIG. 3A presents another embodiment of a tissue-on-a-chip device. The device has a central well in the upper (“apical”) portion, at the bottom of which is disposed a chip having two windows, each of which bears an ultrathin membrane, which is so thin as to be transparent. When media or reagents are added to the well, the membranes in the two windows are fluidly connected by the medium. The bottom of the device of FIG. 3A has a single flow channel, which has two ports on each end allowing fluid introduced through one or both of the two ports on one side at the top of the device to flow through the flow channel and out of the device through either or both of the second ports on the second end of the device. Cells can be positioned in the bottom flow channel under the ultrathin membranes. A photonic integrated circuit (“PIC”) is visible under the upper ultrathin membrane in fluid connection with the cells in the single flow channel. In FIG. 3A, the circles containing the ring sensors photonic ring sensors on the photonic integrated circuit can be seen through the ultrathin membrane. FIG. 3B is a cartoon showing a typical use of the device of FIG. 3A. In this embodiment, tenocytes are cultured in a hydrogel in the lower flow channel on the left side. A dotted lines with an arrowhead represents the diffusion of factors released by the tenocytes into the flow channel, where they can contact the PIC. Factors released by the tenocytes can also diffuse up through the first ultrathin membrane (shown by dashed horizontal lines), across to the second windown and down through the second ultrathin membrane to reach the PIC, but it is expected factors reaching the PIC by that route would be in concentration considerably lower than those reaching the PIC directly from the tenocytes.

FIG. 4. FIG. 4 is a close-up depiction of the two ultrathin membranes exposed in the two windows of a membrane chip. Visible below the membrane are the circles within which are disposed photonic ring sensors forming part of the PIC on the tissue chip.

FIG. 5. FIG. 5 is a depiction of an exemplar embodiment of the invention showing the coupling of a sensor chip from the back side through a microphysiological system (“MPS”). At the top of the MPS, tubes connected to hoses are inserted into the ports on either end of the MPS to introduce media and reagents to the bottom flow channel or channels of the MPS. On the underside of the MPS, a laser provides light to an optical hub, coupling the light to the input coupling grating of a PIC disposed in a flow channel of the MPS.

FIG. 6. FIG. 6 is a graph showing the transmission spectrum of the pairs of sensor rings on an exemplar sensor chip in an MPS. The rings have slightly different radii to separate their transmission spectra.

FIG. 7. FIG. 7 shows, on the top, an assembled “single bottom channel” MPS. On the left, below the assembled device, is an exploded view showing the layers that form the assembled device. Just above the first layer is a depiction of a membrane chip with two windows, each occupied by an ultrathin membrane, that fits into the well of layer L1. The right hand side under the assembled MPS is another exploded view of the layers, this time showing an overhead view of each layer. In between is text stating the material typically used for each layer and the typical thickness of the layer. Not shown is the material used under layer 10 (“L10”) to provide the floor of the MPS, sealing it so that fluid introduced into the flow channel does not exit at the bottom of the device. The floor can be glass, cyclic olefin polymer (COP), or PET. Legend: PET: polyethylene terephthalate, PSA: pressure-sensitive adhesive, PMMA: poly(methyl methacrylate).

FIG. 8. FIG. 8 shows, on the top, an assembled “single bottom channel” MPS. On the left, below the assembled device, is an exploded view showing the layers that form the assembled device. Just above the first layer is a depiction of a membrane chip with two windows, each occupied by an ultrathin membrane, that fits into the well of layer L1. The right hand side under the assembled MPS is another exploded view of the layers, this time showing an overhead view of each layer. In between is text stating the material typically used for each layer and the typical thickness of the layer. Not shown is the material used under layer 10 (“L10”) to provide the floor of the MPS, sealing it so that fluid introduced into the flow channel does not exit at the bottom of the device. The floor can be glass, cyclic olefin polymer (COP), or PET. Legend: PET: polyethylene terephthalate, PSA: pressure-sensitive adhesive, PMMA: poly(methyl methacrylate).

FIG. 9. FIG. 9 presents different configurations of sensor chips usable in the inventive systems and methods.

### DETAILED DESCRIPTION

As noted in the Background, modeling the reaction of human tissues and organs to candidate therapeutic agents has traditionally been performed using cell cultures or animal models. In recent years, pressure to reduce the use of animals in pre-clinical studies and other factors has increased the need for systems, such as organs-on-a-chip (sometimes referred to herein as “OoCs”) and tissues-on-a-chip (sometimes referred to herein as “ToCs”), that can model various aspects of human biology, such as the response of organs and tissues to candidate therapeutic agents. Such model systems are sometimes referred to as microphysiological systems (sometimes abbreviated herein as “MPS”). For convenience, the term “MPS” will be used herein to refer to both OoCs and ToCs, unless one or the other use is specified or required by context. As both OoCs and ToCs have been the subject of much study in the art over at least the past decade, it is expected that the general configuration and features of microphysiological systems such as OoCs and ToCs do not need to be described in detail here.

The present invention provides several improvements to such microphysiological systems, surprisingly improving the ability of the systems to detect factors released by cells or otherwise present in cell culture media or other fluids in which cells in a MPS reside. Some of these improvements relate to using self-contained, on-board sensors that allow MPS devices that allow the presence of analytes present in the cell culture medium to be detected. Some of the improvements relate to the ability to create arrays of MPS with self-contained, on-board sensors that permit high-throughput screening of compounds to determine their effect on cells disposed in the MPS. To avoid confusion, particularly when referring to systems using multiple MPS, MPS themselves will sometimes be referred to herein as “microphysiological system devices” or “MPS devices.”

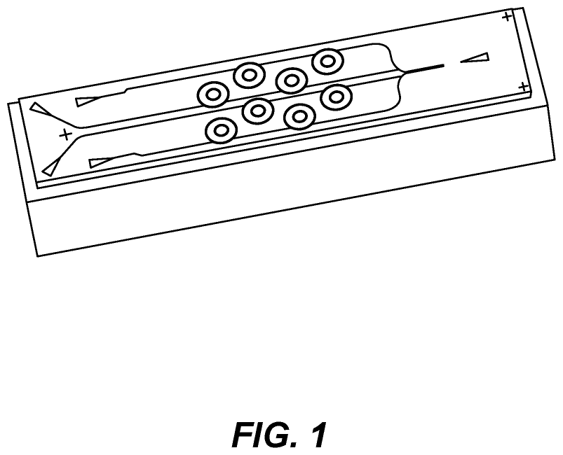

In some aspects, the invention exploits the recent development by some of the present inventors of a sensor platform holding “rice-sized (1×4 mm) silicon nitride ring resonator sensor chips.” See, Cognetti, et al., Lab Chip, 2021, 21:2913-2921; doi.org/10.1039/D1LC00369K, hereafter, “Cognetti 2021.” The contents of Cognetti 2021 are hereby incorporated by reference.) These sensor chips were developed during the pandemic to provide disposable, rapid detection of antibodies to SARS-COV-2. Id. The sensor chips utilize photonic sensors, such as photonic ring sensors, derivatized to detect the presence of one or more analytes of interest if the one or more analytes of interest are present in a solution contacting the photonic sensors. Wave guides connect the photonic sensors to output grating couplers were used that were configured to emit infrared (“IR”) light. FIG. 1 presents a depiction of an exemplar of such a sensor chip, in which photonic ring sensors are disposed in the circles etched in the oxide forming the surface of the silicon-based chip. Each pair of photonic ring sensors is connected by wave guides to an input grating coupler on one side and to an output grating coupler on the other. The rings of the two ring sensors have a different circumference, which causes their resonance to shift, allowing the signal from the two ring sensors to be distinguished. The binding of an analyte of interest to a photonic ring sensor derivatized to specifically bind the analyte causes a change in light emitted by the output grating coupler.

As noted above, the sensor chips used in Cognetti 2021 are 1×4 mm rectangles. References herein to the “sides” of the chips relates to the 1×4 mm rectangular faces (as opposed to the short sides from top to bottom of the chip) unless otherwise specified. One of the rectangular faces, referred to as the “front side” or “front” of the chip, has ring sensors visible, while the other rectangular face, referred to as the “back side,” is featureless when viewed. Referring again to FIG. 1, the ring sensors and wave guides are clearly visible on the front side of the sensor chip.

As persons of skill will appreciate, optical ring resonators are typically coupled from the front of the sensor chip, and detection of IR light from the output grating coupler is likewise detected from the front of the sensor chip. This works well in applications such as the fluidic card used in Cognetti 2021, as the sensor chip was being used to detect the presence or absence of an analyte in a cell-free fluid introduced onto the fluidic card. Due to the configuration of the card and the use for which it was developed, the front side of the card was oriented to face down into the fluid in which the analyte was to be detected. Front side coupling is more problematic for applications in which the sensor chip is used in a microfluidic device (other than a lateral flow device such as a card), in which the sensor chip is typically positioned face up within a plastic or acrylic flow channel, such as in the microfluidic devices shown in co-owned International Application No. PCT/US2021/058498, published as International Publication No. WO 2022/099161.

Surprisingly, we have found that light from the tunable laser can be coupled through the back of the sensor chip, allowing any changes in light from the derivatized photonic ring sensors on the front of the chip to be detected through the back side of the sensor chip, rather than from the front. These findings were unexpected, because the grating couplers used were designed to have a focus point 500 microns off the front of the chip and there was no expectation that the photonic ring sensors could be coupled through the substrate of the back of the sensor chip. It was thus suprising to find that they performed well enough to allow coupling through the back of the chip.

Further surprisingly, we found that we could detect the output of the output grating coupler not only through the substrate of the back of the chip, but also through the bottom layer sealing the microfluidic flow channels of exemplar microphysiological systems in which the sensor chip had been positioned. There was no expectation that the photonic ring sensors could be coupled not only through the substrate of the back of the sensor chip itself, but also through a bottom layer of, for example, glass, plastic or arcylic, sealing the microfluidic flow channel of the microphysiological systems in which the sensor chip was present. So far as we are aware, back side coupling has never before been used for sensing signals from photonic ring resonators indicating the presence of an analyte the photonic ring resonators are derivatized to detect. The output from the output grating couplers was therefore surprisingly more tolerant of variations in distance and materials than expected.

These surprising discoveries have important consequences. First, in previously described MPS, such as those in International Patent Application No. PCT/US2021/058498 (the “'498 PCT application”), the on-board photonic integrated circuits had wires extending from a side of the microfluidic flow device to an external computer to allow transmission of the output of the photonic integrated circuits to the computer for recording and analysis. The discovery that the Cognetti 2021 sensor chips can be coupled from the back side of the chip through one or more layers of a microfluidic flow device such as a microphysiological system (MPS) allows the creation of microfluidic flow devices that are completely sealed, other than for the microfluidic input and output necessary to flow cell culture medium and other reagents over cells within the microfluidic flow device.

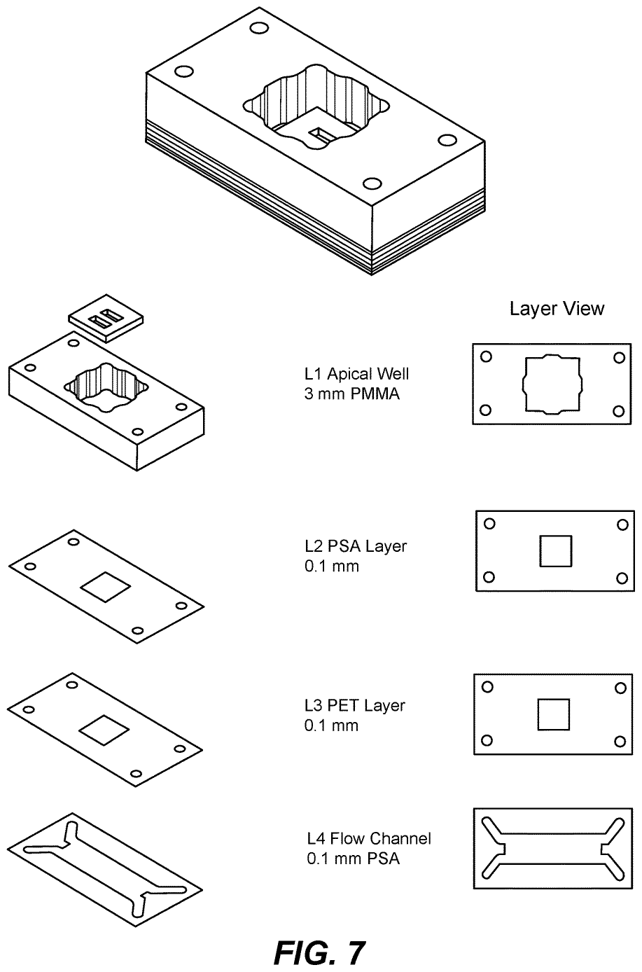

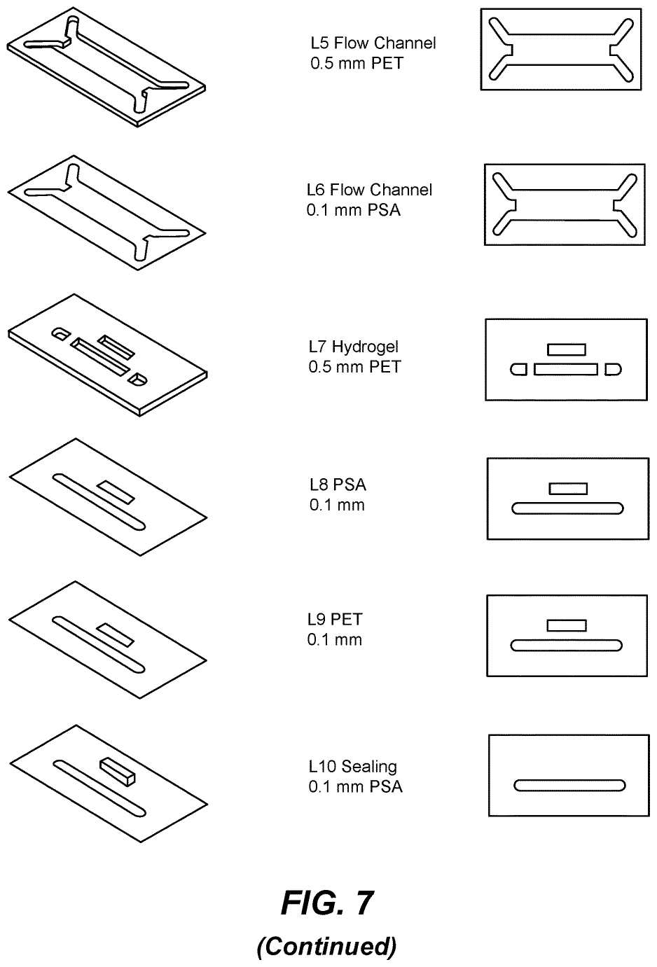

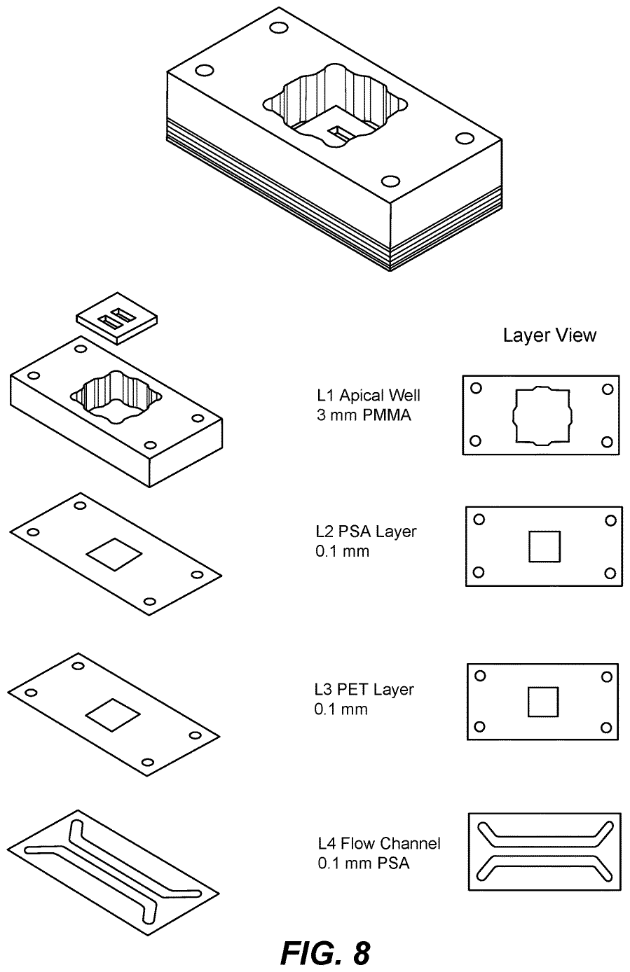

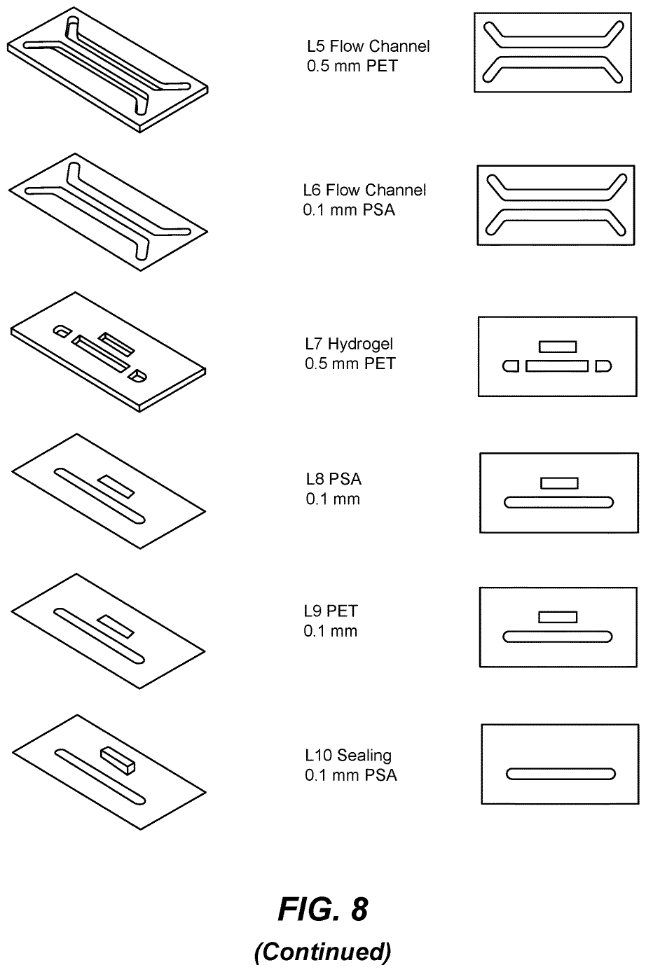

Second, the ability to couple the photonic ring sensor grating couplers through the back side increases flexibility in positioning the sensor chip within an MPS. As persons of skill will appreciate, photonic ring resonators derivatized to detect an analyte of interest in a fluid (such as cell culture medium that has been flowed past cells under study) can only do so if they are in contact with fluid that may or may not contain the analyte. In some prior studies, the physical constraints on positioning in a microfluidic flow device a sensor chip bearing the photonic ring resonators that had to be connected by wires to an external computer could be addressed by positioning the sensor chip face down in the flow channel. Unfortunately, positioning the sensor chip upside down is physically not possible in many, if not all, MPS, due to constraints imposed by the size of the MPS and of the flow channels within the MPS. Thus, the sensor chip often has to be positioned with the front side facing up, where the coupling will have to be done through several layers of material forming the MPS, and often cells growing on a membrane or other surface within the MPS, making interference with the coupling more likely. The ability to couple a sensor chip from the back side, while still sensing the presence of analytes on the front side, allows positioning the sensor chip on a lower flow channel or layer of the MPS, and coupling the grating couplers through fewer layers of the material forming the MPS, and without requiring the light coming into the input grating coupler and that coming out of the output grating coupler to pass through one or more layers of cells within the MPS. In some preferred embodiments, the sensor chip is disposed on the floor of the MPS. Referring to FIGS. 7 and 8, the sensor chip is preferably positioned on the bottom layer of the device, which in FIGS. 7 and 8 is referred to as the so-called “sealing layer,” L10. L10 can be seen to have a flow channel in the layer; the actual sealing of the layer is accomplished by a floor (not shown), typically of glass, cyclic olefin polymer (“COP”), or polyethylene terephthalate (“PET”).

As noted above, previous MPS devices with on-board photonic integrated circuits, such as those described in the '498 PCT application, had wires extending from a side of the MPS device to an external computer. The presence of the wires protruding from the sides of the devices imposed a need for careful alignment of the layers of the devices as they are assembled, so that the very small channels for the wires align almost perfectly. Sensor chips without wires extending from them are sometimes referred to herein as being “self-contained.” MPS devices with self-contained sensor chips allow a much more rapid alignment and assembly of the devices, reduce the complexity of manufacturing, and reduce the cost of manufacturing. Accordingly, the inclusion of photonic integrated circuits that do not need a physical connection to outside equipment is an important advance in making MPSs.

In another aspect, MPS devices with self-contained photonic integrated circuits allows the devices to be positioned edge-to-edge in the same orientation, while MPS devices with external wiring can be placed edge-to-edge only in two rows at a time, and the devices have to be in opposite orientation to one another. Thus, MPS devices with self-contained PICs are more amenable to being provided in closely packed arrays, improving the ability to automate instruments to add cells and media to the MPS devices and to collect IR light from the self-contained photonic integrated circuits. MPS devices with self-contained photonic integrated circuits are therefore particularly suitable for use in high-throughput screening. To avoid confusion with

The dimensions of the MPS with self-contained photonic integrated circuits, and particularly the thickness of the layers and of the overall devices can be varied. Referring to FIG. 7 and FIG. 8, respectively, in some embodiments, the MPS with self-contained photonic integrated circuits has layers corresponding to those shown in FIG. 7 or FIG. 8, respectively, each with a thickness (or height) that is ±20% of that of the corresponding layer shown in the corresponding figure, and the overall device has a thickness that is ±20% of that shown in the corresponding figure (e.g., FIG. 7 or FIG. 8, respectively). In some embodiments, the MPS with self-contained photonic integrated circuits has layers corresponding to those shown in FIG. 7 or FIG. 8, respectively, each with a thickness that is ±15% of that of the corresponding layer shown in FIG. 7 or FIG. 8, respectively,, and the overall device has a thickness that is +15% of that shown in the corresponding figure. In some embodiments, the MPS with self-contained photonic integrated circuits has layers corresponding to those shown in FIG. 7 or FIG. 8, respectively, each with a thickness that is ±10% of that of the corresponding layer shown in FIG. 7 or FIG. 8, respectively, and the overall device has a thickness that is ±10% of that shown in the corresponding figure. In some embodiments, the MPS with self-contained photonic integrated circuits has layers corresponding to those shown in FIG. 7 or FIG. 8, respectively, each with a thickness that is ±5% of that of the corresponding layer shown in FIG. 7 or FIG. 8, respectively, and the overall device has a thickness that is ±5% of that shown in FIG. 7 or FIG. 8, respectively. In some embodiments, the MPS with self-contained photonic integrated circuits has layers corresponding to those shown in FIG. 7 or FIG. 8, respectively, each with a thickness that is that of the corresponding layer shown in FIG. 7 or FIG. 8, respectively, and the overall device has the thickness shown in FIG. 7 or FIG. 8, respectively.

While the layers shown in FIG. 7 are illustrative, persons of skill will recognize the number of layers and their composition will change depending on the particular studies to be conducted using the MPS. It is expected that persons of skill are well familiar with choosing (or designing) MPS with a configuration of layers suitable for the particular study in which they intend to use the MPS. MPSs have flow channels, and the sensor chip holding the photonic integrated circuit (PIC) is preferably placed in the layer forming the bottom of the flow channel. In preferred embodiments, the layer in which the sensor chip is placed is as close as possible to the bottom of the device, with preferably few layers between the PIC and the bottom, to reduce the chance that the layers will interfere with the ability to couple the PIC on the sensor chip to the external light source and equipment for capturiing the output of the output grating coupler. The sensor chip is preferably fixed in position on the layer on which it is placed to make it easier to align equipment external to the MPS, such as the means for providing laser light to the input grating coupler of the PIC. Various means of fixing the sensor chip to the selected layer of the MPS are known in the art, such as using an adhesive to secure the PIC to the selected layer of the MPS, or configuring a layer of the device with an area shaped to hold a portion of the sensor chip, thereby holding it in place. Persons of skill are familiar with placing PICS in microfluidic flow devices and it is assumed they can readily select among the options available to hold the PIC in a fixed position within the MPS.

### Sensor Chips and Coupling of the Sensor Chip

The self-contained sensor chips used in Cognetti 2021 are particularly small and the input coupling grating and the output grating coupler are therefore physically closer together than is common in typical photonic sensor ring assemblies. Lenses can be used to magnify the grating couplers to simplify the process of getting light to the input grating coupler and capturing light from the output grating coupler. Suitable equipment for getting light to and capturing light from small sensor chips is described, for example, in Cognetti 2021 and in Bryan et al., ACS Sens., Feb. 2023, 8(2): 739-747 (“Bryan 2023”). The process of providing light to and capturing light from the grating couplers, and analyzing the light captured from the output grating coupler is standard for using derivatized photonic ring resonators, and it is expected that persons of skill are familiar with adjusting equipment to address the physical characteristics of any particular sensor chip.

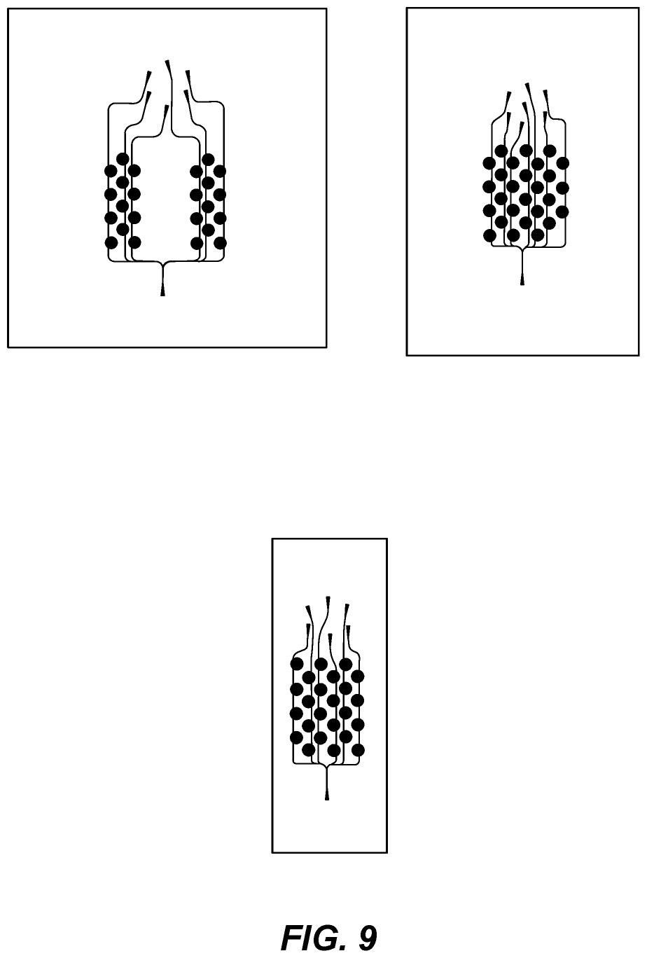

Practitioners that do not want to use lenses to address the proximity of the input grating coupler and the output grating coupler of the Cognetti 2021 sensor chips can use a larger self-contained chip, so long as it fits within the flow channel of the MPS in which it is to be used. Further, the the chips do not have be in the physical configuration of the Cognetti 2021 chips. FIG. 9 presents depictions of the Cognetti chip and of two variations showing chips squarer or broader than the rectangular chip used in Cognetti 2021. Squarer chips may be desirable, for example, in a MPS with a single bottom flow channel. A squarer sensor chip resting on layer L7 will be in contact with more of the culture media and may detect more of a target analyte than a more rectangular sensor chip. Further, other aspects of the PICs can be varied without adversely affecting their function and are within the knowledge and expertise of persons of skill in the art. For example, Cognetti 2021 used silicone nitride waveguides that were 1.5 um wide and 220 nm tall, supporting a single tranverse electric (TE) polarization mode. The waveguides could instead be, for example, 1.2-1.8 μm wide and 180-450 nm tall and could be of a different material known to serve as waveguides. The Cognetti 2021 waveguides were also insulated from the silicon wafer on which they were disposed by a bottom oxide layer 5 μm and a 4 μm cladding was added to isolate the waveguide. Suitable variations would include having a range of insultation and cladding from 2-6 μm.

Persons of skill are familiar with providing laser light to the input grating coupler of photonic ring sensors and of capturing light from the output grating coupler. For example, in Cognetti 2021 describes directing light from a tunable laser source through a polarization controller to obtain linearly polarized light with TE orientation relative to the silicon nitride waveguide.

Light was directed though the input of the optical hub and focused on the input grating of the PIC. Output infrared (IR) light from the output PIC grating was collected by the optical hub and directed through a multimode fiber to an optical power meter, with alignment of the PIC to the optical hub facilitated by a dual-camera VIS/IR microscope. A 5× IR objective lens with on-axis illumination directed light though a long-pass dichroic mirror to either an IR camera or VIS CMOS camera. Proper alignment was confirmed by IR micrograph. The tunable laser and optical power meter were connected to a computer via General-Purpose Interface Bus (GPIB) and controlled by Insertion Loss software of the Keysight Photonic Application Suite (N7700A). Measurements were performed by repeated wavelength scans in the vicinity of resonance signals from the control and probe rings (6 nm scans). As is usual, the resonance redshift is proportional to the binding of the analyte to rings derivatized to detect that analyte. Specific shifts due to capture of the target analyte was calculated by subtracting the redshift of the control ring from that of the probe ring, using a data analysis protocol. Bryan 2023 presents a similar description of how the authors provided light to the input grating coupler of a PIC and captured and analyzed light from the output grating coupler of a PIC. It is expected that persons of skill can adapt the specific methods and equipment described in Cognetti 2021 and Bryan 2023, or similar equipment used in the art for coupling to input grating couplers and coupling to output grating couplers, and analyzing the captured light, to match any particular sensor chip.

Finally, as noted above, that it was a surprise that the Cognetti 2021 sensor chips could be coupled from the back side, given that the grating couplers had been designed to have a focal point 500 micons above the surface. It is expected that the grating couplers of other sensor chips will likewise allow back side coupling with front side sensing. Any particular sensor chip of any particular configuration can, however, be readily tested to see if it is suitable for back side coupling in a particular MPS by simply placing a sample of the sensor chip in question in the bottom layer of a sample of the MPS to be used and seeing if it can be coupled from the back side. Similarly, any given sensor chip of any given configuration can be readily tested to see if it is suitable for placement in a layer of an MPS other than the bottom layer by placing a sample of the sensor chip in question in the desired layer of a sample of the MPS to be used and seeing if the sensor chip can be coupled from the back side.

### Embodiments With a Single Bottom Flow Channel

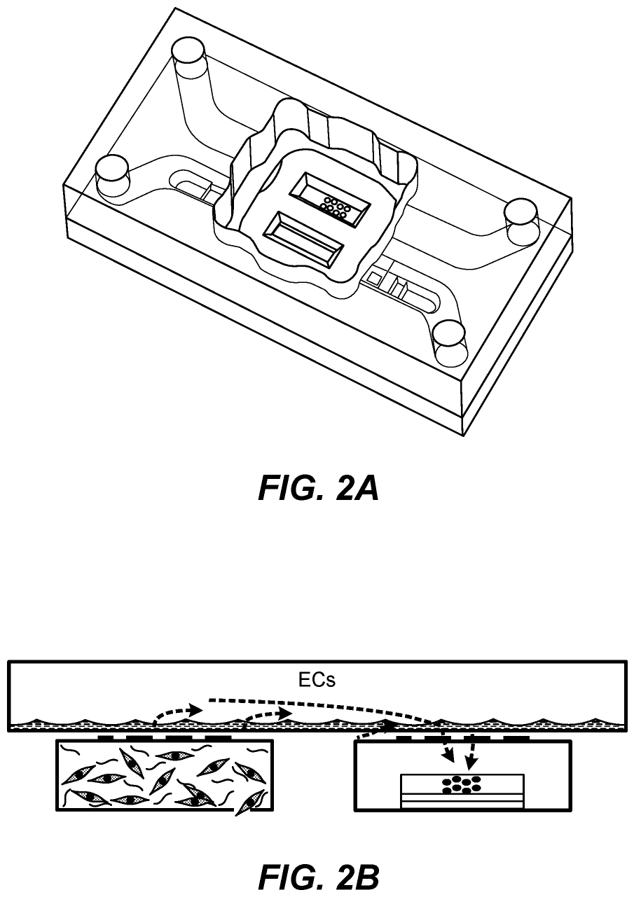

In a first set of embodiments, the invention provides devices configured as shown in FIG. 2A. Proceeding in direction from the top of the device towards the bottom, the device has a top layer which contains an open well. At the bottom of the open well is a holder piece holding an ultrathin membrane, which the holder divides into two slots. Underneath the ultrathin membrane is a bottom chamber, or “flow path,” fluidly connected to ports disposed at the corner, which ports extend through the top layer to allow fluids and cells to be introduced into the bottom chamber and which allow any air in the bottom chamber to exit as fluid is introduced through another port. In a typical embodiment, epithelial cells are grown on the ultrathin membrane, while cells of a tissue type of interest are grown in a portion of the bottom channel. The “single bottom channel” devices of these embodiments can be used to model, for example, the release of cytokines or other factors from cells locally in response to being contacted with chemical factors, such as toxins or therapeutic agents, and thus models the signals are sending to other cells of the tissue around them.

An exemplar use is shown in FIG. 2B. In the exemplar embodiment, epithelial cells (“ECs”) are depicted growing on the upper (apical) side of the slots of the ultrathin membrane (the ultrathin membrane is depicted by horizontal dashed lines), while tenocytes are cultured in a hydrogel in the lower flow channel. Factors released by the tenocytes from the hydrogel (the edge is depicted by a vertical line of dashes) into the flow channel can contact the photonic integrated circuit, as depicted by the generally horizontal line of dots terminating in an arrowhead. Factors released by the ECs can also contact the photonic integrated circuit, as shown by the vertical line terminating with an arrowhead. Factors released by the tenocytes can also diffuse up through the ultrathin membrane, across to the second slot and down into the bottom flow channel, thereby reaching the photonic integrated circuit, but it is expected factors traveling by that route would reach the photonic integrated circuit in concentrations much lower than those reaching the photonic integrated circuit directly from the tenocytes.

In this embodiment, the tenocytes are contained in the hydrogel. Cells of other types can be likewise contained in a hydrogel, or a hydrogel or even a plastic, glass of silicone barrier can be disposed in a portion of the flow channel to prevent the cells from coming into direct contact with the sensor chip.

FIG. 7A presents, on the left side, an exploded view of the layers comprising an exemplar embodiment of a device with a single bottom flow channel. This embodiment is intended for use with tenocytes. Tenocytes are preferably cultured with supports to adhere to in the culture environment, and the design of this exemplar embodiment provides a pair of bars disposed across the width of the channel in layer 7. The cross bars can be omitted in embodiments intended for use in culturing cells that do not grow better when provided with cross supports to which to adhere. Note that the chip holding the ultrathin membrane in the exploded view on the left side is not shown in the middle column but, when the device is assembled, it is positioned on the PET layer that is labeled as layer 3. The middle column also shows the thicknesses of each layer of the MPS device. The right-hand side presents a view of an assembled device.

### Embodiments With Two Bottom Flow Channels

In a second set of embodiments, the devices can be configured with two bottom flow channels, rather than the single channel of the embodiments described above. This second set of embodiments model how cells of the particular tissue in the device communicate with cells of other organs and tissues by releasing factors that reach the systemic circulation.

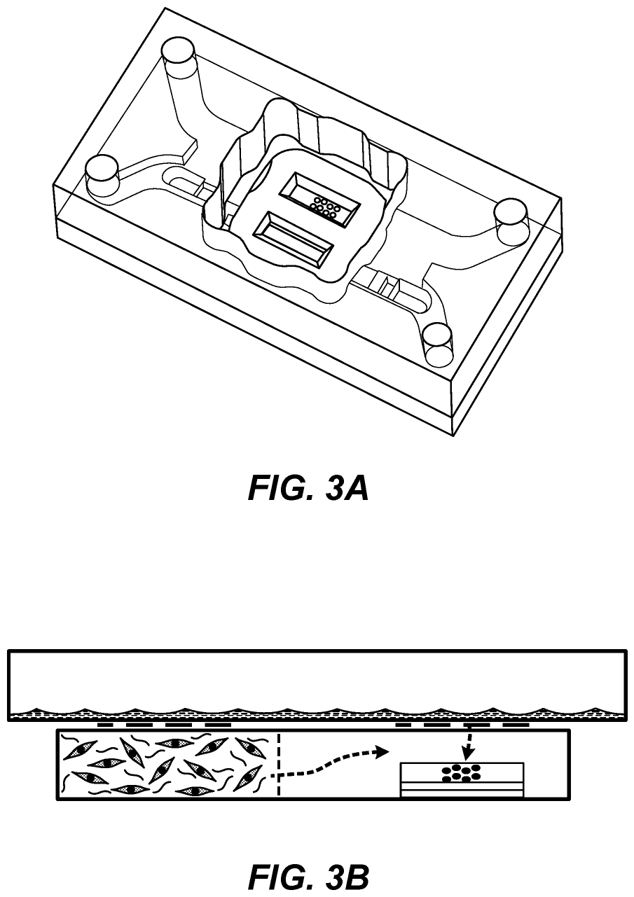

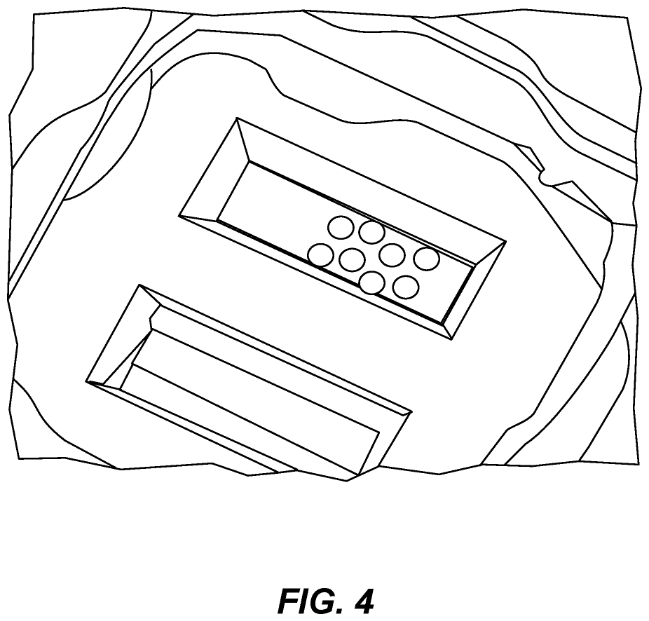

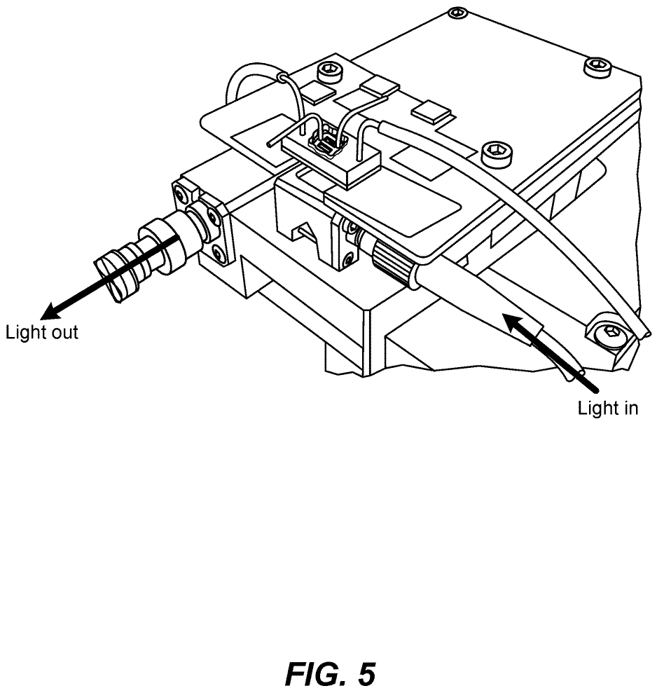

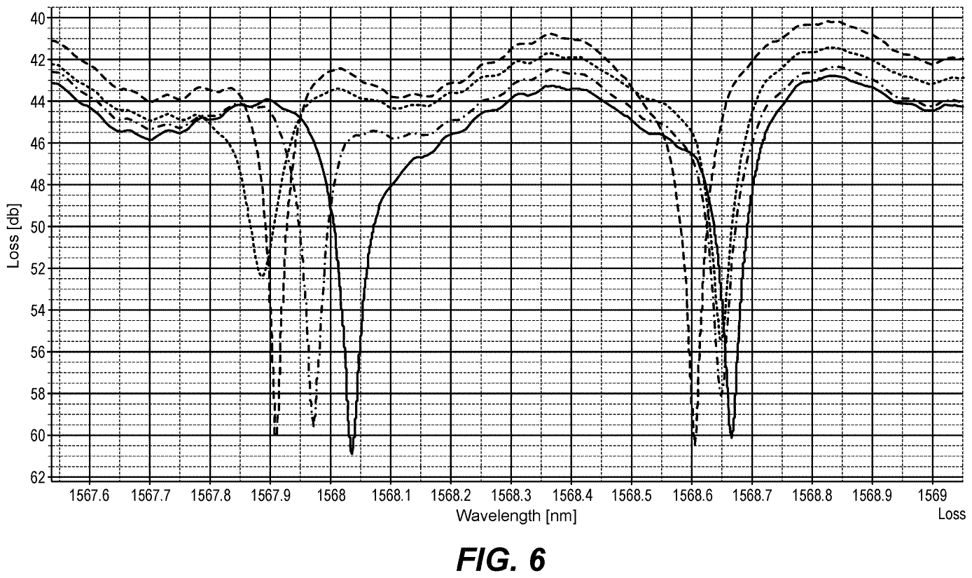

FIG. 3A is a photograph of an exemplar of these embodiments. The device has a central well in the upper (“apical”) portion, in which is disposed an ultrathin membrane held in place by a piece of material, which in this case divides the ultrathin membrane into two slots. When media or reagents are added to the well, the two slots are fluidly connected by the medium. The bottom of the device has two flow channels, one under each slot of the ultrathin membrane. For ease of reference, the two channels will be referred to below as the upper and the lower flow channels, but for clarity, it is noted both the upper and lower flow channels are disposed in the bottom portion of the device, below the ultrathin membrane. Ports for introducing media or reagents into the bottom flow channels, and for allowing air in the channel to exit the device as media or reagents are added, are disposed at the corners of the device and extend through the apical portion of the device, allowing pipettes, tubing, or other conduits of media or other fluids access to the ports from the top of the device. A photonic integrated circuit is disposed in the upper flow channel. The dark circles containing the ring sensors can be seen through the transparent, ultrathin membrane. FIG. 3B presents a cartoon showing a typical use of the device of FIG. 3A. In this embodiment, epithelial cells (“ECs”) are depicted growing on the upper (apical) side of the slots of the ultrathin membrane, which is depicted by horizontal dashed lines, while tenocytes are cultured in a hydrogel in the lower flow channel. Dotted lines with arrowheads depict the diffusion of factors released by the tenocytes from the lower flow channel up through the ultrathin membrane, and through the ultrathin membrane down to the upper flow channel, where they come into contact with the photonic integrated circuit.

FIG. 8A presents, on the left side, an exploded view of the layers comprising an exemplar embodiment of a device with a single bottom flow channel. The embodiment depicted is intended for use with tenocytes. Tenocytes are preferably cultured with supports to adhere to in the culture environment, and the design of this exemplar embodiment provides a pair of bars disposed across the width of the channel in layer 7. The cross bars can be omitted in embodiments intended for use in culturing cells that do not grow better when provided with cross supports to which to adhere. Note that the chip holding the ultrathin membrane in the exploded view on the left side is not shown in the middle column but, when the device is assembled, it is positioned on the PET layer that is labeled as layer 3. The middle column also shows the thicknesses of each layer of the MPS device. The right-hand side shows an assembled device.

### Ultrathin Membranes and Pore Size Ranges

As noted above, the inventive devices comprise a porous ultrathin membrane on which cells can be disposed. Ultrathin (<400 nm thick) precision pore membranes have been made and their properties explored, as exemplified by Striemer, et al., Nature, 2007. 445(7129): p. 749-753; DesOrmeaux, et al., Nanoscale, 2014. 6(18): p. 10798-10805; and Winans, et al., J Memb Sci, 2016. 499: p. 282-289. Ultrathin membranes exhibit a unique combination of filtration properties. They are exceptionally permeable, enabling very low-pressure filtration in microfluidic devices. References to “membranes” in this disclosure refer to ultrathin membranes unless otherwise specified.

Ultrathin membranes have been made using pure silicon, silicon nitride, glass (SiO₂), MgF₂, gold, graphene, and various polymers. Because of their extreme thinness, ultrathin membranes are sometimes referred to as “2D membranes.” It is contemplated that ultrathin membranes made of any of the materials mentioned above can be used in the inventive devices and methods.

In preferred embodiments, the ultrathin membranes are made of silicon, silicon nitride, silicon oxide, or silicon dioxide, as ultrathin membranes made of these materials are particularly robust. In some preferred embodiments, the ultrathin membrane is a silicon nitride ultrathin membrane.

Ultrathin membranes are so thin as to be transparent. They therefore better facilitate use of microscopy to monitor the device, for example to observe cells on the ultrathin membrane.

In some embodiments, the ultrathin membranes have nanopores (“nanoporous membranes”), which for purposes of this disclosure means it has pore sizes of ≤100 nm. In some embodiments, the ultrathin membranes have pores that are mesopores (“mesoporous membranes”), which for purposes of this disclosure means it has pores with sizes >100 nm but <1 μm. In some embodiments, the ultrathin membranes have micropores (“microporous membranes”), which for purposes of this disclosure means it has pore sizes ≥1 μm to 20 μm. In some embodiments, the ultrathin membrane has some pores that are nanopores and some that are mesopores or micropores, while in some embodiments, it may have nanopores, mesopores, and micropores. The pore sizes of ultrathin membranes for use in the inventive devices may be tuned by patterning them with pores of different sizes. For example, some of the pores on the ultrathin membrane may be nanopores and some may be mesopores or micropores. Alternatively, the ultrathin membrane may be provided with some pores that are mesopores and some that are micropores. Ultrathin membranes that are provided with pores of two or all three of the size ranges described above are sometimes referred to herein as “dual-scale membranes.” Selecting the size of the pores on the ultrathin membrane allows better control over the substances that can flow through the pores and, therefore, what reaches the sensor chips. For example, the pore size can be such as to allow analyte diffusion through the membrane, but not cells, or can be sized to allow cells such as monocytes to migrate from one side of the membrane to the other.

### “On-Board” Photonic Integrated Circuit Sensor Chips

The inventive devices and methods utilize photonic integrated circuit sensors integrated into the devices themselves. By designing the devices with the sensors “on-board”, the sensors are much closer to the cells on the ultrathin membrane, allowing them to capture information about the presence or absence of analytes released or passing by the cells (i.e., not taken up by them) than that is both temporally and spatially closer to the cells than allowed by the use of prior chips, which have been positioned “downstream” of the tissue chips or OOCs, and thus further away from the cells of the tissue chip or OOC. Further, previous devices have used electronic sensors, but have not configured or adapted photonic sensors that can read the flow of analytes from tissue chips or OOC. Co-owned International Application PCT/US2021/058498 discloses devices with on-board photonic integrated circuit sensors, but not photonic integrated circuit sensors on the same layer of the device as cells of a tissue or organ of interest, so that the practitioner can choose whether to model factors in the systemic circulation (afforded in the present devices with two bottom flow channels), or factor released locally by the tissue (single bottom flow channel embodiments).

Photonic sensors have numerous advantages over electrical sensors as they do not require redox labels or other reagents to operate, can be fabricated inexpensively at scale using methods developed by the microelectronics and photonics industry, provide sensitive multiplex capability in a very small sensor footprint (down to a few square microns), and employ a measurement geometry in which the light source, sensor element, and detector are all nominally in the same plane, and therefore can be more easily integrated onto layered microfluidic devices than optical sensors incorporating free-space optics.

Ring resonators and photonic crystals use a combination of waveguide and configuration of the waveguide and associated structures that allows modulating the resonance of the sensor. Mach-Zehnder interferometers do not have a “resonance”, but report binding based on a change in phase. In embodiments of the inventive devices, the photonic sensor or sensors are derivatized with a molecule that binds (and preferably, specifically binds) an analyte of interest. For example, the molecule may be an antibody that specifically binds an antigen of interest, such as a particular cytokine or inflammatory biomarker. As another example, the molecule may be a receptor that specifically binds an enzyme that the practitioner wishes to monitor. When the cytokine or enzyme is present and binds to the antibody or the enzyme binds to the receptor, it changes the effective refractive index, signaling that the analyte has been detected. Methods of functionalizing photonic sensors to add antibodies, receptors, or other molecule that functions as an analyte-specific capture probe are known, as exemplified by Mudumba et al., J Immunol Methods, 2017, 448:34-43. An exemplar method is set forth in the Examples.

In a particularly preferred embodiment, the photonic sensors are photonic ring resonator sensors. As known in the art, ring resonator sensors comprise in relevant part a straight waveguide disposed immediately adjacent to a circular waveguide that serves as a ring resonator. Binding of the analyte to the antibody, receptor, or other molecule that functions as an analyte-specific capture probe on or in the immediate proximity of the waveguide changes the index of refraction and changes the resonant wavelengths in the ring resonator. Mudumba et al., supra, note that, as more material is deposited above the ring, the resonant wavelengths shift accordingly. As this shifts the color of the light resonant in the ring, it quantifies the amount of analyte of interest that has bound to the antibody, receptor or other capture molecule on the ring sensor. Changing the diameter of the ring allows the use of different resonance frequencies and makes multiplexing the sensors easy by sensing different analytes by rings of different resonant frequencies.

The small form factor (typically <200 μm diameter) of ring sensors allows for many sensor units to be incorporated on a single chip. Consequently, multiple analytes can be quantified simultaneously. In the sensors described by Mudumba et al., for example, 136 rings, each 30 um in diameter, were etched on a 4×6 mm silicon chip, with 8 covered by a coating as controls and the other 128 rings organized in 32 clusters of 4 functionalized rings each exposed to the material flowing above them. The size of the particular sensors is selected to be suitable according to the desired resonant wavelength, taking into account the minimum bend radius of the particular material chosen for use as the substrate to hold the sensors. The minimum bend radius for materials, such as Si3N4 and Si, commonly used in the art to hold ring resonators, and sizing ring resonators according to the desired resonant wavelength and minimum bend radius, are well known in the art and it is assumed that practitioners can select ring sizes suitable for any particular material they choose to employ as a substrate.

High-Q micro-ring resonators are extremely sensitive to small changes in the refractive index environment above the chip. By modifying the top surface to selectively bind with particular biomolecules, they form the basis for sensing such changes. Working with AIM Photonics (Albany, NY), a national manufacturing institute, we have developed ring resonator designs demonstrating some of the highest sensitivities reported to date (Q=250,000; bulk refractive index sensitivity=253 nm/RIU) (“Q” is a “quality factor,” defined as the wavelength of the resonant signal divided by its linewidth at half-maximum). These devices are readily integrated with microfluidic channels, and, when derivatized with antibodies against proteins of interest, provide sensitive, quantitative detection in complex sample matrices such as serum or cell culture media.

In some embodiments, the devices use photonic sensors other than ring sensors. In some preferred embodiments, the photonic sensors other than ring sensors are photonic crystals. Two-dimensional photonic crystals (sometimes referred to herein as “2DPhCs”) are very small, and potentially allow the detection of single molecules of analytes. See, e.g., Joannopoulos, et al., PHOTONIC CRYSTALS: MOLDING THE FLOW OF LIGHT, 2nd Ed. 2008 (Princeton Univ. Press, Princeton, NJ); Baker et al., Lab on a Chip, 2017, 17:1570-1577; Baker et al., Lab on a Chip, 2015, 15:971-990. In some embodiments, they are spiral wave guides. See, e.g., Densmore et al., Optics Letters, 2008, 33(6): 596-598.doi.org/10.1364/OL. 33.000596. In some embodiments, they are Mach-Zehnder interferometers. See, e.g., Li, et al., Optics Express, 2012, 20(10): 11109-11120.doi.org/10.1364/OE.20.011109.

2DPhCs consist of a periodic array of high refractive index/low refractive index materials. Easily fabricated in silicon-on-insulator (SOI) substrates, 2DPhCs confine light in a very small mode volume, yielding very high sensitivities (single particle) even for devices with a relatively low Q factor, where, as noted above, Q is defined as the linewidth at half height for the primary sensor resonance. Work in the labs of the present inventors has validated 2DPhC devices as highly sensitive sensors for proteins and virus particles in complex sample matrices such as serum. For example, a 2DPhC sensor was designed with several sensor cavitics in series. In this device, introduction of a defect “hole” in the crystal produces a characteristic absorption in the transmission band. Since most of the light passes through the sensor, small variations in the defect size of sensors in series produce absorptions at different wavelengths. Tests done using a device functionalized with an anti-IgG antibody showed it could sense IgG, as a three-fold redundant detector for the protein. Similarly, we demonstrated that this sensor format could specifically detect virus-like particles (VLPs) from human papillomavirus (HPV) doped into fetal bovine serum (FBS). Sensor performance was similar in 10% FBS and buffer; this indicates that the device, coupled with appropriate surface chemistry and blocking methodology, is capable of rejecting nonspecific binding. Finally, a “large defect” structure (where the defect “hole” is matched to the size of a large particle) demonstrated recognition-mediated capture and detection of single particles under fluidic flow. This highlights the exceptional sensitivity of these structures. While photonic crystals are tiny (roughly 10×10 microns), and sensitive to the single particle or (potentially) to the single-molecule level, they are more subject to manufacturing variability than are ring resonators.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A microphysiological system having disposed within it a self-contained photonic integrated circuit sensor chip.

2. The microphysiological system of claim 1, which microphysiological system is an organ-on-a-chip.

3. The microphysiological system of claim 1, which microphysiological system is an tissue-on-a-chip.

4. The microphysiological system of claim 1, which system has a first compartment, which first compartment comprises a first flow channel, wherein said self-contained photonic integrated circuit sensor chip is positioned in said first flow channel.

5. The microphysiological system of claim 4, wherein said self-contained photonic integrated circuit sensor chip is fixed in position in said first flow channel.

6. The microphysiological system of claim 5, wherein said self-contained photonic integrated circuit sensor chip is fixed in position in said first flow channel by an adhesive.

7. The microphysiological system of claim 5, wherein said self-contained photonic integrated circuit sensor chip is fixed in position in said first flow channel by a layer of said microphysiological system shaped to fit around a portion of said self-contained photonic integrated circuit sensor chip.

8. The microphysiological system of claim 1, wherein said first compartment further comprises a second flow channel.

9. The microphysiological system of claim 2, further comprising a second compartment, which second top compartment is positioned over said first compartment.

10. The microphysiological system of claim 7, wherein a first ultrathin membrane is positioned between said second compartment and said first compartment.

11. The microphysiological system of claim 8, further wherein a second ultrathin membrane is positioned between said second compartment and said first compartment.

12. The microphysiological system of claim 8, wherein said first ultrathin membrane is positioned between said second compartment and said first compartment over said self-contained photonic integrated circuit sensor chip.

13. The microphysiological system of claim 8, wherein a plurality of cells of a first cell type are seeded on said ultrathin membrane, and a plurality of cells of a second cell type are seeded in said first flow channel.

14. A system for studying an analyte of interest released from cells in a microphysiological system device, said system comprising
(a) a microphysiological system device having a top and a bottom and having disposed within it a self-contained photonic integrated circuit sensor chip (“chip”) having photonic ring sensors, waveguides, an input grating coupler and an output grating coupler, said chip having a front side and a back side, wherein said front side of said chip is closer to said top of said device than is said back side of said chip, and
(b) an external apparatus for (i) coupling light from a laser to said input grating coupler of said self-contained photonic integrated circuit sensor chip through the back side of said chip and (ii) coupling light from said output grating coupler of said self-contained photonic integrated circuit sensor chip through the back side of said self-contained photonic integrated circuit sensor chip.

15. The system of claim 14, further wherein said photonic ring sensors of self-contained photonic integrated circuit sensor chip have been derivatized to detect said analyte of interest.

16. The system of claim 14, wherein said microphysiological system device is a tissue-on-a-chip.

17. The system of claim 14, wherein said microphysiological system device is an organ-on-a-chip.

18. A method for studying an analyte of interest released from cells in a microphysiological system device, said method comprising
(1) providing (a) a microphysiological system device having a top and a bottom and having disposed within said device a self-contained photonic integrated circuit sensor chip (“chip”), said chip having a front side and a back side, wherein said front side is closer to said top of said device than said back side, and having photonic ring sensors, waveguides, an input grating coupler and an output grating coupler, which photonic ring sensors have been derivatized to detect said analyte of interest, and
(b) an external apparatus for (i) coupling light from a laser to said input grating coupler of said chip through the said back side of said chip and (ii) coupling light from said output grating coupler of said chip through said back side of said chip,
(2) growing cells in a fluid in said microphysiological system device, and
(3) contacting said derivatized photonic ring sensors with said fluid in which said cells have been grown,
whereby a change in light from said derivatized photonic ring sensors indicates the presence of said analyte of interest.

19. An array of at least two microphysiological system devices, each having disposed within said them a self-contained photonic integrated circuit sensor chip.
